(19) United States
(12) Patent Application Publication
(10) Pub. No.: US 2006/0240560 A1
(43) Pub. Date: Oct. 26, 2006

(54) Microsphere optical ion sensors based on doped silica gel templates

(76) Inventors: Eric Bakker, West Lafayette, IN (US); Chao Xu, West Lafayette, IN (US); Katarzyna Wygladacz, Lafayette, IN (US)

Correspondence Address: SHELDON & MAK PC, 225 SOUTH LAKE AVENUE, 9TH FLOOR, PASADENA, CA, 91101, US

(21) Appl. No.: 11/345,553

(22) Filed: Jan. 31, 2006

Related U.S. Application Data
(60) Provisional application No. 60/648,527, filed on Jan. 31, 2005.

(51) Int. Cl. G01N 33/20

(52) U.S. Cl. 436/84

(57) ABSTRACT

A sensor for determining the concentration of a target ion in a liquid sample having a particulate silica doped with: an ionophore capable of binding the target ion; and an indicator capable of producing a detectable signal in response to binding by the ionophore of the target ion. The detectable signal is related to the target ion concentration in the liquid sample.

### CROSS REFERENCE TO RELATED APPLICATION

The present application claims the benefit of priority to U.S. Provisional Patent Application No. 60/648,527, filed on Jan. 31, 2005, the entire contents of which are hereby incorporated herein by reference.

### STATEMENT REGARDING FEDERALLY SPONSORED RESEARCH OR DEVELOPMENT

This invention was made with United States Government support under DE14950 awarded by the National Institutes of Health. The United States Government has certain rights in the invention.

### BACKGROUND

The present invention relates to microsphere-based chemical sensors and, more particularly, to microsphere optical ion sensors based on doped silica templates.

As part of a concerted effort in sensor miniaturization, the fabrication of microsphere-based chemical sensors and biosensors has gained increased interest in the past decade. There are several advantages of a microsphere-sensing platform. The micrometer scale of the sensors enables interrogation of analyte concentrations in a defined local environment, such as in single cells. Additionally, the required sample volumes are much smaller, which can increase sensitivity, shorten response time, lower the associated cost of reagents, and improve the lower detection limit. Moreover, reading out a great number of identical microspheres improves precision because of the high redundancy of the sensing information. Microsphere-based sensing principles have been successfully applied to a range of readout formats, such as optical imaging fibers and flow cytometry, such explained in for example, U.S. Patent Publication No. 2004/0058384 entitled “Ion-Detecting Microspheres And Methods Of Use Thereof, the entire contents of which are hereby incorporated herein by reference.

Among the various analytical assays that can be incorporated into microspheres, neutral-carrier-based microsphere optodes offer the ability to reliably measure the ion activities of common electrolytes. Ion or molecule-sensing microspheres have been prepared by various means, including polymer swelling as described in W. Seitz et al., Anal. Chim. Acta 400 (1999) 55; and Z. Shakhsher et al., Microchim. Acta.144 (2004) 147. Microspheres have also been prepared by heterogeneous polymerization as described in S. Peper et al., Anal. Chim. Acta 442 (2001) 25, surface adsorption involving particle templates as described in S. Shibata et al., Jpn. J. Appl. Phys. 37 (1998) 41, and by solvent casting as described in I. Tsagkatakis et al., Anal. Chem. 73 (2001) 315.

A sonic casting device, similar to that disclosed in U.S. Pat. No. 4,162,282, has been constructed for the mass production of optical sensing microspheres with controllable size under mild, non-reactive conditions as explained in I. Tsagkatakis et al., Anal. Chem. 73 (2001) 6083. The optical ion-sensing microspheres fabricated were found to obey classical bulk optode theory and were used for measurements of Na⁺, K⁺, Ca²⁺, Pb²⁺, and Cl⁻. More recently, plasticizer-free microspheres based on a methylmethacrylate-decylmethacrylate (MMA-DMA) copolymer matrix were developed for K⁺ using a particle casting device in an effort to circumvent plasticizer leaching problems as explained in S. Peper, A. Ceresa, Y. Qin, E. Bakker, Anal. Chim. Acta 500 (2003) 127.

However, the above methods suffer from one or more of the following shortcomings. The equipment necessary to produce the microspheres requires specialized mechanical parts and accessories that are not available to most research labs. The generated microspheres are suspended in water, which may not be suited for all applications. The life times of the microspheres have been limited to less than 6 months and more typically from 2-6 weeks. These earlier particles have poor mechanical stability; they may break apart with sonication, producing fines (fragments) that interfere with measurement. Additionally, these prior particles may coalesce with one another or with container walls. This coalescence is more likely if the particles are stored at high local concentrations as commonly occurs when particles sediment during storage.

Silica particles have been widely used as the stationary phase to pack chromatography columns. The silica surface may be chemically modified to suit the need for chiral separations as explained in W. Pirkle et al., J. Org. Chem. 44 (1979) 1957; and N. Oi et al., J. Chromatogr. 292 (1984) 427. Additionally, the silica surface may be coated with a suitable polymer to fabricate a stationary phase with optimal separation properties as explained in H. Figge et al., J. Chromatogr. 351 (1986) 393.

Doped silica particles with dyes have been used for vapor sensing as explained in K. J. Albert et al, Anal. Chem. 72 (2000) 1947, or as biomolecular markers as explained in Y. Qin et al. Anal. Chem. 75 (2003) 3038. However, most of the uses of doped silica particles were in only one-component sensing systems and are therefore not suitable for the purpose of ion sensing.

Therefore, a need exists for improved ion-sensing microspheres.

### SUMMARY

Accordingly, the present invention is directed to a sensor for determining the concentration of a target ion in a liquid sample, the sensor comprising: a particulate silica doped with an ionophore capable of binding target ions in the sample and an indicator capable of producing a detectable signal in response to binding by the ionophore of the target ion. The detectable signal is related to the ion concentration in the liquid sample. The indicator can be a chromoionophore.

The sensor can also have a self-plasticizing polymer. Optionally, the sensor includes a supporting polymer and a plasticizer. The supporting polymer can be PVC and the plasticizer can be bis(2-ethylhexyl) sebacate (DOS).

The particulate silica can have a spherical or other three dimensional shape. Optionally, the particulate silica is silanized. The sensor can also include a lipophilic cation exchanger. The lipophilic cation exchanger can be sodium tetrakis[3,5-bis(tri-fluoromethyl)-phenyl]borate (NaTFPB).

The present invention is also directed to a method of detecting an ion in a liquid sample using the sensors. Optionally, sensors made from silica gel microspheres containing water can be dried to produce dried sensors for storage until use. The dried sensors can be resuspended to produce resuspended sensors and used for detection. Optionally, the sensors can be passed through a flow cytometer for measuring the detectable signal.

The sensors can also be used in an optical fiber bundle.

### BRIEF DESCRIPTION OF THE DRAWINGS

These features, aspects and advantages of the present invention will become better understood with regard to the following description, appended claims and accompanying drawings where:

FIG. 1a is a scanning electron micrograph of silanized silica particles useful in the present invention before doping with sensing ingredients;

FIG. 1b is a scanning electron micrograph of the particles of FIG. 1a following doping with sensing ingredients of Example Na-J;

FIG. 2 is a three-dimensional plot of spatially resolved fluorescence spectra observed from a single silica-based Na⁺-selective microsphere optical sensor of example Na-J in contact with (A) 10⁻²M HCL and (B) 10⁻²M NaOH;

FIG. 3a is a plot illustrating the response of microspheres according to example Na-J at pH 7.4 as characterized by fluorescence microscopy;

FIG. 3b is a plot illustrating the response of microspheres according to example Ca-L at pH 7.4 as characterized by fluorescence microscopy;

FIG. 4 is a table showing the experimental selectivity coefficients for optodes containing various ionophores normalized to pH 7.4;

FIG. 5a is a plot illustrating the response of microspheres according to example Na-J at pH 7.4 as characterized by analytical flow cytometry;

FIG. 5b is a plot illustrating the response of microspheres according to example Ca-L at pH 7.4 as characterized by analytical flow cytometry;

FIG. 6 is a photomicrograph of the observed spatial coverage of Na⁺-selective microspheres according example Na—H on etched wells of an optical fiber bundle; and

FIG. 7 is a three-dimensional plot of the fluorescence spectra of five neighboring Na⁺-selective microspheres according to example Na—H on etched wells of an optical fiber bundle.

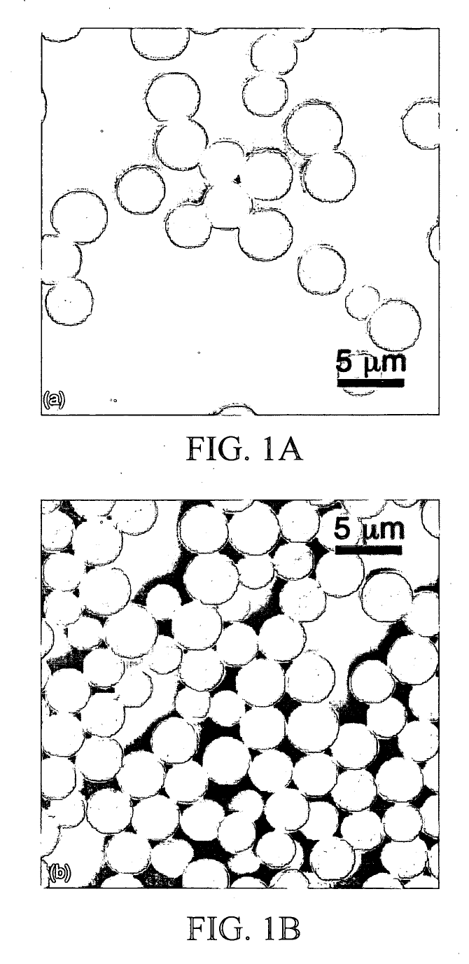

### DETAILED DESCRIPTION

The present invention, according to an embodiment, is directed to ion-selective optical sensors based on doped particulate silica templates and methods for making and using them. The present invention is also the subject of an article entitled “Microsphere Optical Ion Sensors Based On Doped Silica Gel Templates,” in Analytica Chimica Acta, 537, 29 Apr. 2005, pp. 135-143, the entire contents of which are hereby incorporated herein by reference.

Preferably, the sensors are fabricated from microspheres having a porous silica substrate. In an embodiment of the present invention, the silica substrate is Kromasil 100 Å spherical silica with a mean particle size of about 3.5 μm from EKA Chemicals, Sweden. Among others, additional silica substrates that may be used with the present invention include, other spherical silica with reasonably tight size distributions, for example Kromasil 100 Å in diameters of 5, 7, 10, 13, or 16 μm. The size of the microspheres may range from about 0.2 μm to about 50 μm, and preferably range from about 0.5 μm to about 20 μm.

The sensors have an ionophore capable of binding to, and having high selectivity for, target ions in a liquid sample. The sensors may be used in connection with a wide variety of ionophores for detecting different target ions. Examples of such ionophores include, but are not limited to, ionophores selective for target ions such as hydrogen, Li⁺, Na⁺, K⁺, Ca²⁺, or Mg²⁺, or metal ions such as Pb²⁺, Cu²⁺, Hg²⁺, Ag⁺, and oxides such as UO₂²⁺.

In an embodiment of the present invention, the ionophore was tert-butylcalix[4]arene tetraethyl ester (sodium ionophore X). In another embodiment of the present invention, the ionophore was a Ca²⁺ionophore AU-1 grafted in poly(n-butyl acrylate). The concentration of ionophore can be from about 0.1 to about 200 mmoles/kg, and preferably from about 10 to about 50 mmole/kg.

Additional ionophores that may be used with the present invention include, for example, Potassium Ionophore I, (Valinomycin), Potassium Ionophore II (Bis[(benzo-15-crown-4)-4′-ylmethyl]pimelate), Potassium Ionophore III (BME 44; [2-Dodecyl-2-methyl-1,3-propanediyl-bis [N-(5′-nitro(benzo-15-crown-5)-4′-yl)carbamate]], Chloride Ionophore I (5,10,15,20-Tetraphenyl-21H,23H-porphin manganese(III) chloride; Mn(III)TPPCl), Chloride Ionophore II (ETH 9009; [4,5-Dimethyl-3,6-dioctyloxy-1,2-phenylen]-bis-(mercury-trifluoroacetate), Sodium Ionophore I (ETH 227; N,N′,N″-Triheptyl-N,N′,N″-trimethyl-4,4′,4″-propylidynetris(3-oxabutyramide)), Sodium Ionophore II, (ETH 157; N,N′-Dibenzyl-N,N′-diphenyl-1,2-phenylenedioxydiacetamide), Sodium Ionophore III (ETH 2120; N,N,N′,N′-Tetracyclohexyl-1,2-phenylenedioxydiacetamide), Sodium Ionophore IV (DD-16-C-5, 2,3:11,12-Didecalino-16-crown-5), Sodium Ionophore V (ETH 4120; 4-Octadecanoyloxymethyl-N,N,N′,N′-tetracyclohexyl-1,2-phenylenedioxydiacetamide), Sodium Ionophore VI(Bis[(12-crown-4)methyl] dodecylmethylmalonate), Sodium Ionophore VIII (Bis[(12-crown-4)methyl] 2,2-didodecylmalonate), Sodium Ionophore X (4-tert-Butylcalix[4]arene-tetraacetic acid tetraethylester), Calcium Ionophore I (ETH 1001; (−)-(R,R)-N,N′-(Bis(11-ethoxycarbonyl)undecyl)-N,N′-4,5-tetramethyl-3,6-dioxaoctanediamide; Diethyl N,N′-[(4R,5R)-4,5-dimethyl-1,8-dioxo-3,6-dioxaoctamethylene]-bis(12-methylamino-dodecanoate), Calcium Ionophore II (ETH 129; N,N,N′,N′-Tetracyclo-3-oxapentanediamide), Calcium Ionophore III (A 23187; Calcimycin), Calcium Ionophore IV (ETH 5234; N,N-Dicyclohexyl-N′,N′-dioctadecyl-3-oxapentanamide) all of which are available from Fluka (Milwaukee, Wis.).

The sensors also comprise an indicator capable of producing a detectable signal in response to binding by the ionophore of the target ion. In an embodiment of the present invention, the indicator is a chromoionophore. The chromoionophore allows for quantitation and/or detection of target ions in the sample. Deprotonation of the chromoionophore occurs when protons are exchanged by target ions binding with the ionophore, and changes in chromoionophore protonation result in measurable changes in its optical behavior.

The chromoionophore can be for example, 9-(diethylamino)-5-octadecanoylimino-5H-benzo[a]phenoxazine (chromoionophore I, ETH 5294). Additional indicators that may be used with the present invention include, for example, Chromoionophore II; ETH 2439; 9-Dimethylamino-5-[4-(16-butyl-2,14-dioxo-3,15-dioxaeicosyl)phenylimino]benzo[a]phenoxazine, Chromoionophore VI; ETH 7075; 4′,5′-Dibromofluorescein octadecyl ester, and Chromoionophore III; ETH 5350; 9-(Diethylamino)-5-[(2-octyldecyl)imono]benzo[a]phenoxazine.

The sensors can also comprise a self-plasticizing polymer such as poly(n-butyl)acrylate or a copolymer of methyl methacryate (MMA) and decyl methacrylate monomers as described in U.S. patent application Ser. No. 10/313,090, filed on Dec. 5, 2002, the entire contents of which are hereby incorporated herein by reference.

Additionally, the sensors can include a supporting polymer and a plasticizer. The supporting polymer can be, for example, high-molecular-weight poly(vinyl chloride) (PVC). The plasticizer can be, for example, bis(2-ethylhexyl) sebacate (DOS) from Fluka (Milwaukee, Wis.). Additional plasticizers include Bis(2-ethylhexyl)phthalate and 2-Nitrophenyl octyl ether.

The sensors of the present invention may also include other additives, such as ion-exchangers, to enhance the extraction of the target ion from the sample and the migration of the target ion to the ionophore. Preferably, the ion-exchanger is a lipophilic cation exchanger. The lipophilic cation exchanger can be, for example, sodium tetrakis[3,5-bis(tri-fluoromethyl)-phenyl]borate (NaTFPB) from Dojindo Molecular Technologies, Inc., USA.

Other useful cation exchangers include carbaclosododecaborates, particularly halogenated carborane anions. Examples of halogenated dodecacarborane cation exchangers include trimethylammonium-2, 3, 4, 5, 6, 7, 8, 9, 10, 11, 12 undecabromocarborane (TMAUBC) (see U.S. patent application Ser. No. 10/313,090), and salts (e.g., trimethylammonium salts) of undecachlorinatedcarborane (UCC), hexabrominatedcarborane (HBC) and undecaiodinatedcarborane (UIC) anions.

Preparation of microspheres according to an embodiment of the present invention will now be described. Silanization can be performed prior to doping to increase the hydrophobicity of the silica particles according to K. Wygladacz et al., Sens. Actuators B 83 (2002) 109; and M. E. McGovern et al., Langmuir (1994) 360, the entire contents of which are hereby incorporated herin by reference. Preferably, the silica templates are carefully sealed in a bottle, or other container, and kept under vacuum to remove air from pores. The silica particles are then doped with appropriate sensing ingredients.

The sensing ingredients, including the ionophore and the indicator, are dissolved in a suitable solvent, such as tetrahydrofuran (THF) and mixed gently with the silica templates. The mixture is then covered, for example with aluminum foil, and then preferably kept in the dark until the sensing ingredients are introduced into the porous silica templates upon evaporation of the solvent. Preferably, the fabricated microspheres are kept dry and in darkness before use.

Preferably, the microsphere optical sensors are doped with a cation-exchanger (“R−”), an ionophore (“L”) and an H⁺-selective chromoionophore (“Ind”). For such bulk optodes, the cationic analytes (“Iz+”) with a charge of (“z+”) are extracted from the aqueous solution into the organic sensing phase, thereby expelling hydrogen ions via the following cation-exchange mechanism:
I^{z+}(aq)+nL(org)+zIndH⁺(org)+zR⁻(org)=Lₙ^{z+}(org)+zInd(org)+zR⁻(org)+zH⁺(aq) (1)

Combining charge and mass balances and defining the mole fraction of protonated “Ind” as (“1−α”), the activity of the analyte may be expressed as:

$${a}_{1} = {( {z}_{1} {K}_{ex} )}^{- 1} {( \frac{α}{α - 1} {a}_{H} )}^{{Z}_{I}} \times \frac{{R}_{T}^{-} - ( 1 - α ) {Ind}_{T}}{{\{ {L}_{T} ( {R}_{T}^{-} - ( 1 - α ) {Ind}_{T} ) ( {n}_{I} / {z}_{I} ) \}}^{{n}_{I}}} \qquad ( 2 )$$

In a buffered solution where the pH is known, the activity of analyte ion can be determined by the degree of protonation of the chromoionophore (“1−α”), which is calculated based on the observed emission intensities for the protonated (“Rpro”) and unprotonated form (“Rdep”) of the chromoionophore:

$$α = \frac{[ Ind ]}{{Ind}_{T}} = 1 + {( \frac{{R}_{pro} - R}{R - {R}_{dep}} )}^{- 1} \qquad ( 3 )$$

As will be discussed below in regard to the examples, fluorescence microscopy and flow cytometry measurements show that the responses of the microsphere optical sensors of the present invention obey classic bulk optode theory. The measuring ranges are compatible with physiological electrolyte levels and the obtained selectivity data are comparable with those of silica-free sensing particles. Additionally, the microsphere optical sensors of the present invention have shelf lives of more than 6 months if stored in dry form.

### EXAMPLES

Preparation of the Ca²⁺ ionophore AU-1

The Ca²⁺ ionophore N,N-Dicyclohexyl-N′-phenyl-N′-3-(2-propenoyl)oxyphenyl-3-oxapentanediamide (AU-1) was synthesized according to the method described in Y. Qin, S. Peper, A. Radu, A. Ceresa, E. Bakker, Anal. Chem. 75 (2003) 3038, the entire contents of which are incorporated herein by reference, and grafted in poly(n-butyl acrylate) at 2% (w/w) and 5% (w/w) as described below. N-Butyl acrylate was obtained from Polysciences (Warrington, Pa.).

Briefly, the AU-1 was synthezied using the following method. Step 1: To a stirred solution of diglycolic anhydride (1.16 g, 10 mmol) in 100 mL of dry dichloromethane was added dicyclohexylamine (3.62 g, 20 mmol). The mixture was stirred at room temperature for 3 h. Then, 20 mL of 6 N HCl was added to the reaction mixture. The solid was filtered, and the organic layer of the filtrate was separated and dried with anhydrous sodium sulfate. Dichloromethane was removed using a rotary evaporator. White crystals of 3-oxapentane acid-N,N′-dicyclohexylamide (I) were recrystallized from ethyl. acetate in 92% yield (2.74 g). Step 2: 3-Hydroxyldiphenylamine (3.33 g, 18 mmol) was dissolved in 25 mL of dry THF with N₂ protection. Subsequently, triethylamine (1.98 g, 19.5 mmol) was added to the solution. Afterward, acryloyl chloride (1.62 g, 18 mmol) was added dropwise to the reaction mixture with a syringe under N₂ at −5° C. After 25 min, 30 mL of a saturated NaHCO₃ solution was added to quench the reaction. The organic phase was then separated and washed with water. After evaporation of the solvent, the crude product was purified by flash chromatography (1:1 EtOAc/hexane). A pale yellow solid, m-anilinophenyl acrylate (II) was obtained in 60% yield (2.88 g). Step 3: To a solution of I (0.736 g) and II (0.529 g) in 30 mL of dry CH₂Cl₂ was added Et₃N (0.8 g) at room temperature while stirring. Then, 0.612 g of BOP-CL was added. The mixture was refluxed for 24 hours. The reaction mixture was washed with 10 mL of saturated NaHCO₃ and water. The organic phase was obtained after separation and evaporation of the solvent. The leftover was purified using flash chromatography (1:5 EtOAc/hexane). A pale yellow solid (C₃₁H₃₈N₂O₅, MW 518.65) was obtained in 50% yield.

The polymers incorporating AU-1 were synthesized via thermally initiated free radical solution polymerization. Ethyl acetate solutions containing n-butyl acrylate (1 g) and appropriate amounts of ionophore AU-1 (2 or 5 wt. %) were purged with N₂ for 10 min before adding 5.1 mg of a polymerization initiator azobis-(isobutyronitrile), 98% (AIBN), from Aldrich (Milwaukee, Wis.). The homogeneous solution was continuously stirred and the temperature was ramped to 90° C., which was maintained for 16 hours.

After the reaction was complete, the solvent was evaporated and the polymer was re-dissolved in 10 mL of dioxane. Aliquots of polymer solution (2 mL) were added to 100 mL of distilled water under-vigorous stirring. The white precipitate was collected and dissolved in 25 mL of dichloromethane, followed by water removal with anhydrous Na₂SO₄ and filtering. The solvent was evaporated and the resultant transparent polymer was dried under ambient laboratory conditions. Two batches with different concentrations of ionophore AU-1 grafted in poly(n-butyl acrylate) were synthesized: (1) AU-1 at 2 wt. % (38.6 mmol/kg) and (2) AU-1 at 5 wt. % (96.5 mmol/kg).

Preparation of Microspheres

Several different types of microspheres were made using the following method. Silanization was performed prior to doping. Kromasil 100 Å spherical silica particles with a mean particle size of about 3.5 μm were washed with toluene to remove impurities, connected to vacuum to remove air, and mixed with 3-(trimethoxysilyl)propylmethacrylate (10%, v/v, in toluene) in a flat-bottomed reactor. The temperature was kept at 60-70° C. for 3-4 hours with water reflux. Subsequently, excessive reagents and solvent were removed and the silanized microspheres were washed and continuously connected to vacuum.

The silanized silica templates were then doped with appropriate sensing ingredients at a total mass of 20 mg (doping ingredients+silica templates). The silica templates were carefully sealed in a bottle and kept under vacuum before and after weighing to remove air from pores. The sensing ingredients were dissolved in THF and mixed gently with the silica templates. The mixture was covered with 155 aluminum foil and kept in the dark for 72 h. The sensing ingredients were introduced into the porous silica templates upon evaporation of the solvent during this time. The fabricated microspheres were kept dry in darkness before characterization.

Sodium-Selective Microspheres

Types Na-A to Na-E consisted of 40 mmol/kg sodium ionophore (X), 10 mmol/kg ETH 5294, 20 mmol/kg NaTFPB and various contents of DOS (10, 20, 30, 40, 50%, w/w), mixed with an appropriate amount of silica templates (17.1, 15.1, 13.1, 11.1 or 9.1 mg), respectively. Modified compositions (also with a 20-mg total mass) consisted of the same concentrations as above for sodium ionophore (X), ETH 5294 and NaTFPB, except that DOS was replaced with either 5 or 10% poly(n-butyl acrylate) (types Na—F and Na-G) or 5 wt. % PVC (type Na—H) or (5 wt. % PVC+10 wt. % DOS) (type Na—I), respectively. Type Na-J contained 39.3 mmol/kg sodium ionophore (X), 9.7 mmol/kg ETH 5294, 19.1 mmol/kg NaTFPB, 2 wt. % PVC and 10 wt. % DOS with 17.6 mg silica templates (total mass 20 mg).

Calcium-Selective Microspheres

Microspheres Doped With The Ionophore Ca (IV)

Types Ca-A, B, C, and D were doped using N,N-dicyclohexyl-N′,N′-dioctadecyl-3-oxapentane amide (Ca ionophore IV, ETH 5234) (10.9, 21.5, 39.0 or 48.9 mmol/kg), ETH 5294 (2.0, 4.1, 5.0 or 6.2 mmol/kg), NaTFPB (3.4, 6.0, 7.5 or 9.1 mmol/kg) and 10% (w/w) DOS, with 16.0, 15.5, 15.1 and 14.7 mg silica templates, respectively.

Types Ca-E to Ca-G contained 39.0 mmol/kg Ca (IV) ionophore, 5.0 mmol/kg ETH 5294, 7.5 mmol/kg NaTFPB, combined with either 5 or 10 wt. % poly(n-butyl acrylate) (types Ca-E and Ca—F) or with 5 wt. % PVC (type Ca-G).

Microspheres Doped With AU-1 Ionophore Grafted To poly(n-butyl acrylate).

Types Ca—H to Ca—K had 2 wt. % AU-1 grafted in poly(n-butyl acrylate). Types Ca—H to Ca—K had 15, 30, 40 or 50% (w/w) of polymer to the total mass, which translated into 5.8, 11.6, 15.4 and 19.3 mmol/kg of AU-1; 0.6, 1.2, 3.0 or 3.6-mmol/kg of ETH 5294; 1.1, 2.3, 4.6 or 5.8 mmol/kg of NaTFPB; and 10 wt. % DOS with 17.4, 14.3, 9.7 or 8.0 mg of silica templates, respectively.

Type Ca-L had 5 wt. % AU-1 grafted in poly(n-butyl acrylate). AU-1 has recently been grafted into an MMA-DMA copolymer matrix for the fabrication of plasticizer-free ion-sensing systems such as ion-selective membranes and thin optode films. See, Y. Qin, S. Peper, A. Radu, A. Ceresa, E. Bakker, Anal. Chem. 75 (2003) 3038. Poly(n-butyl) acrylate, was previously reported by Heng and Hall as a useful internally plasticized polymer, L. Heng, E. Hall, Anal. Chem. 72 (2000) 42. Type Ca-L had 16 wt. % of polymer (30.1 mmol/kg Ca²+ionophore AU-1), 4.2 mmol/kg ETH 5294, 8.0 mmol/kg NaTFPB and 10 wt. % DOS doped into 11.9 mg silanized silica templates.

Example Testing

Fluorescence microscopy was performed on a PARISS Imaging Spectrometer (Light Form, Belle Mead, N.J.) in combination with a Nikon Eclipse E400 microscope [15]. The system was equipped with two EDC 1000L CCD cameras (Electrim Corp., Princeton, N.J.) and an epifluorescence mercury lamp (Southern Micro Instruments, Ga.), in addition to a motorized stage (Prior Optiscan ES9, Fulbourn, Cambs, U.K.) manipulated by the Pariss spectral imaging software (Light Form). For characterization and resolving spectra of the fabricated microspheres, a Nikon Plan Fluor 40×0.75 objective was used in combination with an EX510-560 nm filter. The exposure time was chosen from 200 to 600 ms for satisfactory fluorescence intensities.

Microspheres were equilibrated in buffer sample solutions and kept in the dark for 20-40 min. Ten millimoles of HCl or 10 mM NaOH was used to record the spectra at the state of full protonation or deprotonation, respectively. Six to ten microspheres were randomly chosen to record the spectra. The degree of protonation was obtained by calculating the ratio of the two fluorescence intensity peaks of ETH 5294 at 645 and 675 nm.

Flow cytometry experiments were carried out with a Beckman Coulter EPICS XL flow cytometer modified by replacing the standard laser with a 635 nm diode laser and providing filters and detectors selected to measure fluorescence in the wavelength range of 650-675 nm. Fluorescence emitted between 650 and 675 nm was collected with a 650 nm long-pass emission filter and a 660 (±15)-nm band pass filter. The silica-gel-based microspheres were immersed in buffer sample solutions for 20-30 minutes to equilibrate.

A Zeiss DSM 940 scanning electron microscope was used at 5 kV to obtain the SEM images of the silica templates and doped microsphere sensors in the manner detailed in I. Tsagkatakis et al., Anal. Chem. 73 (2001) 6083, the entire contents of which is hereby incorporated herein by reference. Before measurements, dry microspheres were deposited onto an aluminum stub and sputter-coated with 10-20 nm of Au/Pd for about 60 seconds.

Sample Buffer Solutions

All buffer solutions were prepared in Nanopure-deionized water (18.2 MΩcm). An amount of 10⁻⁵ to 1M NaCl or CaCl₂ were prepared respectively in 10⁻³M Tris (tris[hydromethyl]amino-methane from Fluka (Milwaukee, Wis.)) or 10⁻²M MES and pH was adjusted to 7.4 for Tris (or 5.5 for MES). The separate solution method was applied for selectivity measurements as detailed in M. Lerchi et al., Anal. Chem. 64 (1992) 1534; and E. Bakker et al., Anal. Chem. 64 (1992) 1805, the entire contents of which are hereby incorporated herein by reference.

Selectivity determination of bulk optodes requires a complete calibration curve for every ionic species involved. These curves are ideally obtained at a uniform pH for all species and with the same membrane composition. The separate calibration gives the most exact information about the behavior of all analyte ions with respect to thermodynamic exchange and coextraction constants and stoichiometry.

The individual response curves of the ions leading to the selectivity coefficients of the primary over any interfering ion can be plotted graphically and calculated with equations 27 and 36 from E. Bakker et al., Anal. Chem. 64 (1992) 1805.

The response was recorded in 10⁻³M Tris buffers at pH 7.4 containing 1M of one interfering ion salt. For Na⁺-selective microspheres based on silica templates, the measured interfering cations were K⁺, Mg²⁺ and Ca²⁺, while for Ca²⁺-selective microspheres, K⁺, Na⁺ and Mg²⁺ were measured.

Testing Results

Na⁺-Selective Microspheres

The type Na-A microspheres made using the plasticizer DOS were qualitatively responsive to variations in Na⁺ activities. However, deviations between particles and from the theoretically expected response behavior were quite large, and after 24 hours, leaching of the plasticized components was detected under the microscope. In alternate compositions Na—B to Na-E, all of which utilized the plasticizer DOS in different concentrations, the level of leaching actually increased with increasing plasticizer content.

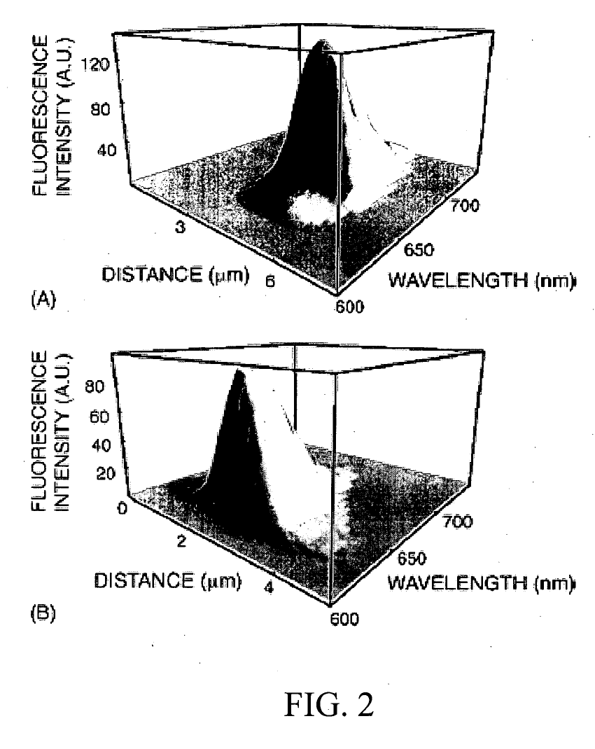

The results from types Na—F to Na-J, where the plasticizer DOS was replaced with poly(n-butyl acrylate) or when PVC was added to the cocktail formulation, showed that adding PVC, as in type Na-J, best improved the response characteristics of the fabricated microspheres. FIG. 2 illustrates the 3D response spectra of type Na-J silica-based Na⁺-sensing microspheres, with the dye in its fully protonated (10⁻²M HCl) and deprotonated forms (10⁻²M NaOH), both of which showed peak shapes similar to those of PVC-based microspheres.

This confirmed the basic functionality of the chromoionophore after doping into the silica templates. The dye ETH 5294 (Chromoionophore I) is an H⁺-selective chromoionophore with dual fluorescence emission maxima at 645 nm (deprotonation) and 675 nm (protonation) in doped silica templates. By taking the intensity ratios of the two peaks, the degree of protonation of the chromoionophore was calculated with Eq. (2). A ratiometric measurement is advantageous for achieving a reliable signal with reduced risk of photo-bleaching and less influence from the light source instability and the size variance of microspheres.

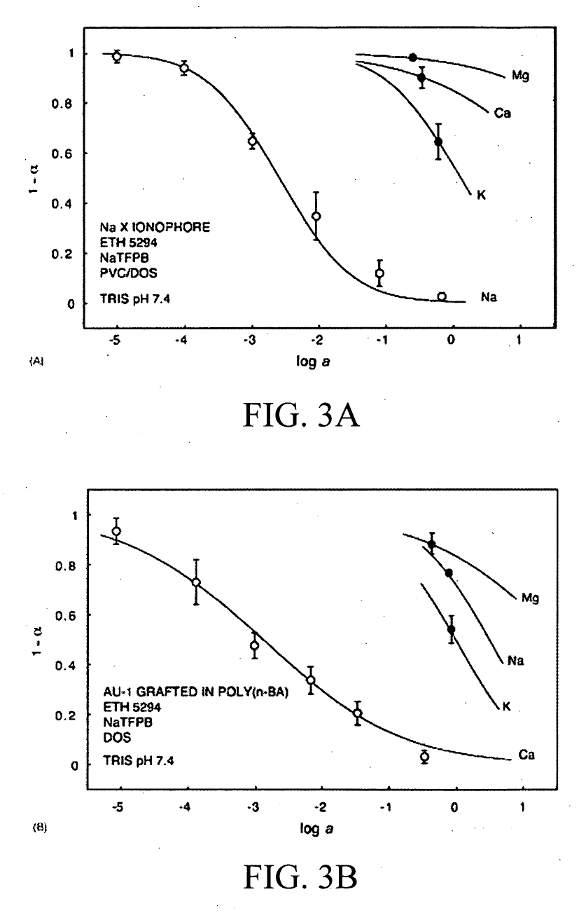

FIG. 3A shows the corresponding Na⁺ response curve together with the associated selectivity of type Na-J microparticles at pH 7.4 as characterized by fluorescence microscopy. The plotted data points are mean experimental values, and error bars indicate the observed standard deviations from 5 to 10 individual measurements.

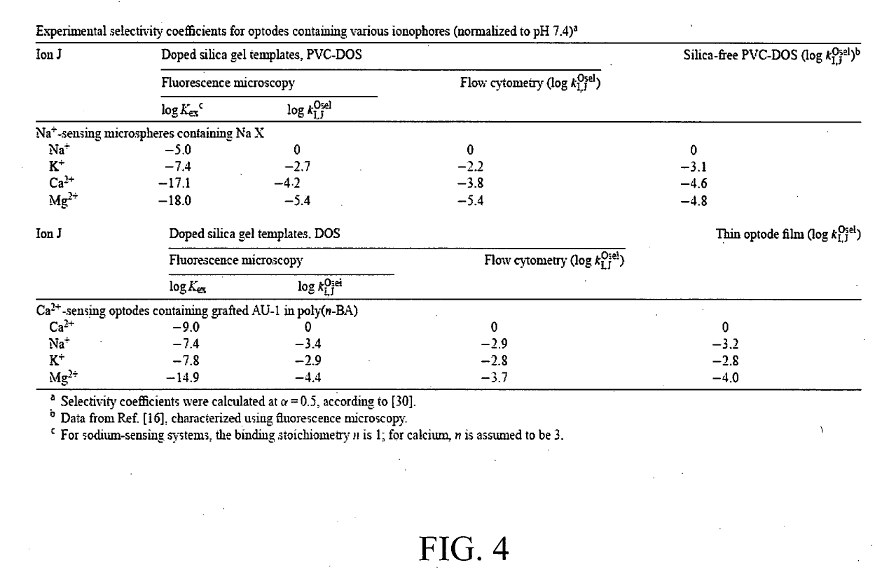

The theoretical curve was derived from Eq. (2) using the experimental composition. The appropriate ion-exchange constants (Kex in Eq. (2)) for the theoretical curves and selectivity coefficients for different sensing systems toward common interfering ions are summarized in the table of FIG. 4 and are compared with data from silica-free PVC-DOS particles made with a sonic particle-casting instrument.

The microspheres of the present invention have approximately the same selectivities toward K⁺, Ca²⁺, and Mg²⁺ as silica-free PVC-DOS particles made with a sonic particle-casting instrument. For the microspheres fabricated from silica templates, the measuring range is suitable for direct measurements of human saliva (stimulated, pH 7.0-7.5, Na⁺ typically 4.3-28 mM). The microspheres fabricated from silica templates can also be used to measure 10-fold diluted human blood plasma (Na⁺ 135-150 mM at pH 7.4).

Ca²⁺-Selective Microspheres

No appropriate calcium response was observed in the microspheres of types Ca-A to Ca-D containing the ionophore Ca (IV), ETH 5294, NaTFPB and DOS in varying compositions. In types Ca-E to Ca-G, which used alternate matrices, including poly(n-butyl acrylate) or which added PVC, the observed calcium response was still unsatisfactory.

In microspheres of types Ca—H to Ca—K, which used AU-1 grafted to poly(n-butyl)acrylate at (2%, w/w), it was found that the resulting functional concentration of the chromoionophore was too low for reliable fluorescence microscopy. A further increase of the concentration of grafted ionophore resulted in strongly aggregating microspheres.

In microspheres of type Ca-L, where the concentration of AU-1 in the poly(n-butyl)acrylate polymer was 5% (w/w), successful selectivity for Ca²⁺ was observed. FIG. 3B shows the Ca²⁺ response observed for type Ca-L at pH 7.4 with the theoretical calibration curve according to Eq. (2).

The observed selectivity of type Ca-L microspheres to common interfering cations in physiological samples was in accordance with data obtained from thin optode films (see FIG. 4). The selectivity over Na⁺, an important interference because of its abundance, was over three orders of magnitude.

At half protonation of the chromoionophore (α=0.5), the corresponding Ca²⁺ activity at pH 7.4 was ˜1 mM, indicating that the measuring range is suitable to directly determine Ca²⁺ in human plasma (1-1.2 mM) at pH 7.4, or stimulated human saliva (0.8-2.8 mM) at a pH of 7.0-7.5.

An equilibration time of about 10 min was typically observed for fabricated microspheres based on doped silica gel templates, which is slightly longer than with regular plasticized PVC particles, but shorter than MMA-DMA based particles. The calcium-selective optical-sensing microspheres doped with grafted AU-1 exhibited longer equilibration times (about 25 min) than the sodium-selective microspheres using a freely dissolved ionophore.

Flow Cytometry Analysis Of Microspheres

Flow cytometry is suitable for characterization of fluorescent microsphere optical sensors based on plasticized PVC, where a single-parameter histogram of the deprotonated form of the chromoionophore ETH 5294 was recorded to determine fluorescence change. Both flow cytometry and fluorescence microscopy were applied to the characterization of the fabricated microspheres. While flow cytometry is not able to spatially or spectrally resolve the fluorescence of individual particles as in fluorescence microscopy, it may provide information on the statistical behavior of a great number of particles.

In a histogram from the FL1 channel which has a 650 nm long pass filter followed by a 660-nm band-pass filter (±15 nm), the number of observed counts was plotted versus a logarithmic value of the peak channel fluorescence and a Gaussian-shaped curve was generated from the accumulated particle counts. As the sample ion concentration increased, the fluorescence intensity from the deprotonated form of ETH 5294 was found to increase as well and resulted in a peak shift of the Gaussian curve, reflecting the ion response. The coefficient of variation (CV) of the entire histogram from about 10,000 microspheres ranged from 7.13 to 29.33, which was larger than observed in regular PVC particles by sonic casting, suggesting poorer size reproducibility, limited by the size distribution of the original silica gel templates.

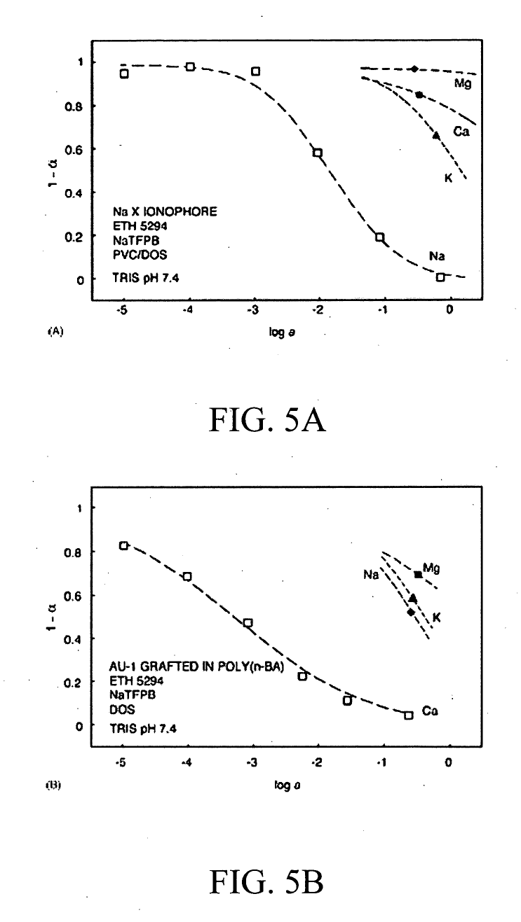

FIGS. 5A and 5B show the calibration curves and associated selectivity data of the sodium (type Na—H) or calcium-selective (type Ca-L) microspheres obtained from flow cytometry measurement. Assuming optode behavior of the fabricated microspheres, the degree of protonation (“1−α”) was described by the fluorescence peak position (P) in the single-parameter histogram from FL1 channel with Eq. (4):

$$α = 1 + {( \frac{{P}_{pro} - P}{P - {P}_{dep}} )}^{- 1} \qquad ( 4 )$$

For the theoretical curve (Eq. (2)) for the sodium response in FIG. 5A, logKₑₓ was found as −5.6, and for calcium, log Kₑₓ=−9.1. These data are similar to those obtained from fluorescence microscopy on individual particles (logKₑₓ=−5.0 for Na⁺ and −9.0 for Ca²⁺, see FIG. 4). The experimental selectivity coefficients (log k^{Osel}_{I,J} values) for potential interferences are also shown in FIG. 4. The measuring ranges were also close to those obtained from fluorescence microscopy. Overall, the response and selectivity data obtained from flow cytometry and microscopy corresponded to each other well, and both techniques were suitable to characterize the microspheres of the present invention.
Application of Silica Based Microspheres to Fiber Optic Sensor Arrays

Regular PVC optical ion-sensing microspheres have been successfully applied to optical fiber bundles that can achieve parallel examination on the responses of tens of thousands of sensors. Optical fiber bundles have been explored as a platform for this purpose, mainly because they can be simply etched to create highly uniform “wells” that fit the size of fabricated microsphere sensors. By immersing the sensing end of the optic fiber bundle in the analyte buffer solutions of different concentrations, the fluorescence spectra the chromoionophore can be captured from the other end of the optical fiber bundle and characterized by fluorescence microscopy. On an optical fiber bundle with a diameter of 2 mm, there are about 3500 individual fiber threads with core diameters of about 4.6 μm.

Plasticized PVC microspheres selective to different ions have been randomly deposited on the same optical fiber bundle to achieve multiple optical sensing. As an alternative to plasticized PVC microspheres, ion-sensing microspheres based on doped silica gel particles according to the present invention can be deposited on the etched distal end of an optical fiber bundle.

A hexagon optical fiber bundle was polished, cleaned, etched and sonicated according to methods known in the art, such as explained in J. R. Epstein et al., Biosens. Bioelectron. 18 (2003) 541, the entire contents of which are hereby incorporated herein by reference. Fabricated Na⁺-selective microspheres according to example Na—H were mixed with deionized water and a 1 μL aliquot of the suspension mixture was placed on the etched well end of the fiber bundle. After the microspheres settled in the wells, deionized water was used to wash off excessive particles. Subsequently, the etched end of the optical fiber bundle was immersed in 10⁻²M HCl for 20 min before the spectral response was acquired.

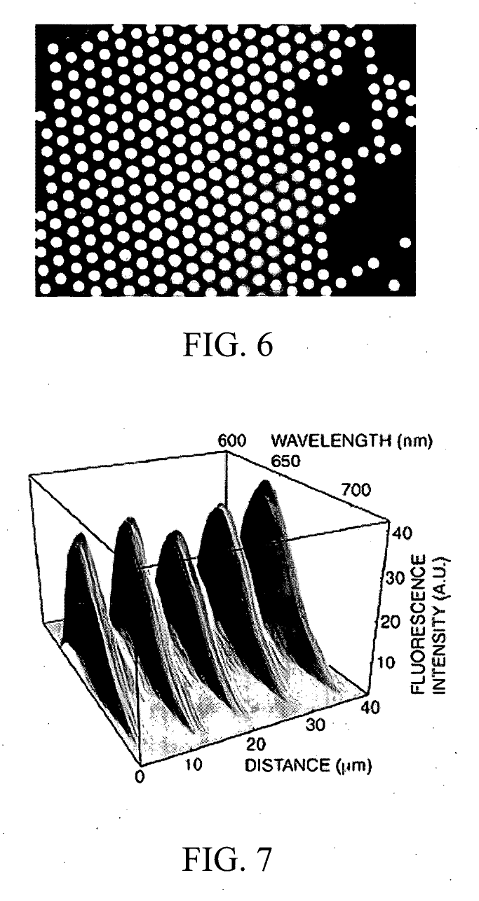

A 90% particle coverage on the optical fiber bundle was observed in fluorescence mode as shown in FIG. 6. The diameters of the fabricated microspheres (˜3.5 μm) are suitable for the size of the etched wells (˜4.6 μm). FIG. 7 presents an observed three dimensional fluorescence spectral image of five Na⁺-selective microspheres (of example Na—H) found in a single line in the etched wells of the optical fiber bundle. The identical shape and close intensity values indicate a good reproducibility of the fluorescence spectra among nearby microspheres.

The microspheres according to the present invention satisfy a number of criteria for successful use in physiological samples, including a reliable ion response and selectivity toward common interfering ions. The presence of the silica template does not appear to influence the sensing chemistry, and the responses of the microspheres reflect the sensing principle of bulk optodes. Detected responses are comparable to those obtained from thin optode films and sonic cast polymeric microspheres.

Additionally, the microspheres of the present invention do not require a curing process as in the case of regular PVC-based microspheres. After doping, the microspheres of the present invention may be used immediately. Because of their high density, microspheres of the present invention can be centrifuged and easily handled either dry or in aqueous solutions.

When sealed and kept dry in darkness, the microspheres of the present invention can be kept for more than 6 months. The microspheres can then be resuspended to produce a resuspended composite. Flow cytometry measurements were repeated 6 months after doping for micro-spheres of type Na—H and Ca-L, and the resulting responses were found to reproduce the initial measurements.

Having thus described the invention, it should be apparent that numerous modifications and adaptations may be resorted to without departing from the scope and fair meaning of the instant invention as set forth hereinabove and as described hereinbelow by the claims.

Although the present invention has been described in considerable detail with reference to certain preferred versions thereof, other versions are possible. Therefore, the spirit and scope of the appended claims should not be limited to the description of the preferred versions described herein.

All features disclosed in the specification, including the claims, abstracts, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means” for performing a specified function or “step” for performing a specified function, should not be interpreted as a “means” or “step” clause as specified in 35 U.S.C. §112.

What is claimed is:

1. A sensor for determining the concentration of a target ion in a liquid sample comprising a particulate silica doped with:
an ionophore capable of binding the target ion; and
an indicator capable of producing a detectable signal in response to binding by the ionophore of the target ion;
wherein the detectable signal is related to the target ion concentration in the liquid sample.

2. The sensor of claim 1 wherein the indicator is a chromoionophore.

3. The sensor of claim 1 wherein the ionophore is tert-butylcalix[4]arene tetraethyl ester.

4. The sensor of claim 1 wherein the sensor further comprises a self-plasticizing polymer.

5. The sensor of claim 4 wherein the self-plasticizing polymer is poly(n-butyl acrylate).

6. The sensor of claim 4 wherein the ionophore is AU-1 grafted into the self-plasticizing polymer.

7. The sensor of claim 1 wherein the sensor further comprises a supporting polymer and a plasticizer.

8. The sensor of claim 7 wherein the supporting polymer is PVC and the plasticizer is DOS.

9. The sensor of claim 1 wherein the particulate silica is a spherical particle.

10. The sensor of claim 1 wherein the particulate silica is silanized.

11. The sensor of claim 1 wherein the sensor further comprises a lipophilic cation exchanger.

12. The sensor of claim 11 wherein the lipophilic cation exchanger is NaTFPB.

13. A method of detecting a target ion in a liquid sample, the method comprising the steps of:
a) exposing a plurality of the sensors of claim 1 to the liquid sample thereby allowing the ionophore to bind the target ion in the sample; and
b) measuring the detectable signal.

14. The method of claim 13 wherein the step of measuring the detectable signal comprises passing the sensors through a flow cytometer.

15. A method of making sensors for determining an ion concentration in a liquid sample comprising the steps of:
a) obtaining a plurality of silica gel microspheres, the silica gel microspheres containing water;
b) doping the microspheres with an ionophore capable of binding ions in the sample and an indicator capable of producing a detectable signal in response to ion binding by the ionophore to create a suspension of sensors;
c) drying the sensors to remove substantially all of the water.

16. The method of claim 15 further comprising the step of resuspending the dried sensors to produce resuspended sensors.

17. An apparatus for determining an ion concentration in a liquid sample comprising:
a) an optical fiber bundle having a plurality of fibers, each fiber having a sample end with a well; and
b) a plurality of microparticles, each microparticle comprising: a silica substrate doped with an ionophore capable of binding ions in the sample; and an indicator capable of producing a detectable signal in response to ion binding by the ionophore;
wherein the detectable signal is related to the ion concentration in the liquid sample; and
the plurality of microparticles are positioned in the wells of the fibers.

18. A sensor for determining an ion concentration in a liquid sample prepared by the method of claim 15.

19. A sensor for determining an ion concentration in a liquid sample prepared by the method of claim 16.
